(19) United States
(12) Patent Application Publication
(10) Pub. No.: US 2015/0223422 A1
(43) Pub. Date: Aug. 13, 2015

(54) NOVEL PLANT VARIETY BRASSICA CAMPESTRIS L. SSP. CHINENSIS HONGKYEONGCHAE, AND METHOD FOR BREEDING THE SAME

(71) Applicant: Sung Ha LEE, (US)

(72) Inventors: Kwan Ho Lee, Suwon-si (KR); Kue Hyon Hong, Suwon-si (KR); Young Hoe Woo, Incheon (KR)

(73) Assignee: Sung Ha LEE, Yangpyeong-gun, Gyeonggi-do (KR)

(21) Appl. No.: 14/425,597

(22) PCT Filed: Sep. 4, 2013
(86) PCT No.: PCT/KR2013/007965

§ 371 Date: Mar. 3, 2015

(30) Foreign Application Priority Data
Sep. 4, 2012 (KR) 10-2012-0097532

(51) Int. Cl. A01H 5/10; A01H 1/02

(52) U.S. Cl. CPC A01H 5/10; A01H 1/02

(57) ABSTRACT

The present invention provides novel Brassica campestris L. ssp. chinensis Hongkyeongchae, and a seed thereof by abandoning an interspecific hybrid F₁ of Brassica campestris var. chinensis and Brassica koreana Lee var. redleaf for the stabilization of a hybrid embryo; back crossing a F₂ plant by using Brassica campestris var. chinensis as a seed parent; obtaining various strains of plants having the shape of conventional Brassica campestris var. chinensis and showing red color; obtaining a plurality of red Brassica campestris var. chinensis from the progenies obtained by back crossing various strains of B₁F₁ plants selected from the obtained plants with Brassica campestris var. chinensis; and carrying out strain acclimation and a self-incompatibility test, a seed productivity test and a local adaption test of a remarkable strain of a plant having the shape of conventional Brassica campestris var. chinensis and showing red color.

### TECHNICAL FIELD

The present invention relates to a novel plant Brassica campestris L. ssp. chinensis Hongkyeongchae, and method for breeding the same and more particularly relates to a novel method for breeding of a novel plant Brassica campestris L. ssp. chinensis Hongkyeongchae by abandoning an interspecific hybrid F₁ of Brassica campestris var. chinensis and Brassica koreana Lee var. redleaf for the stabilization of a hybrid embryo; back crossing a F₂ plant by using Brassica campestris var. chinensis as a seed parent; obtaining various strains of plants having the shape of conventional Brassica campestris var. chinensis and showing red color; obtaining a plurality of red Brassica campestris var. chinensis from the progenies obtained by back crossing various strains of B₁F₁ plants selected from the obtained plants with Brassica campestris var. chinensis; and carrying out strain acclimation and a self-incompatibility test, a seed productivity test and a local adaption test of a remarkable strain of a plant having the shape of conventional Brassica campestris var. chinensis and showing red color.

### BACKGROUND ART

Brassica campestris var. chinensis is a vegetable belonging to Brassicaceae family with short growth period and its leaf and leafy are close to each other. Particularly, when the whole vegetable is green, it is called “Chungkyeongchae” and when the leafy is white, it is called “Baekkyeongchae”. Although it is originated from China, nowadays it is one of the world-wide food materials and which grown in the vinyl green house has tender leaves and easy to take and contains lots of carotene which relates to intake of Vitamin C and Vitamin A.

In addition, Brassica koreana Lee var. redleaf is a novel plant which is produced by heteroploidy breeding of interspecific hybrid of a cabbage and a Chinese cabbage. The Brassica koreana Lee var. redleaf is not suitable for leaf warp because the leaf shape is oval and the stem is stiff and also it is not suitable for salad because the chewy texture is not smooth. Therefore, the Korean Patent Publication No. 10-2008-0091586 disclosed a novel plant for leaf wrap, green vegetable juice or salad which has resolved the above mentioned problems.

Accordingly, the present inventors tried to produce a novel plant having the botanical characteristics of Brassica campestris var. chinensis which has tender leaves and suitable for leaf wrap and the botanical characteristic of Brassica koreana Lee var. redleaf which has red color simultaneously.

### TECHNICAL PROBLEM

Accordingly, the object of the present invention is to provide a novel plant, Brassica campestris L. ssp. chinensis Hongkyeongchae for leaf wrap, green vegetable juice, salad and Kimchi with Chungkyeongchea-shaped and red color by breeding Brassica campestris var. chinensis and Brassica koreana Lee var. redleaf.

Another object of the present invention is to provide a method for breeding of a novel plant, Brassica campestris L. ssp. chinensis Hongkyeongchae and the seed thereof.

### TECHNICAL SOLUTION

The object of the present invention is achieved by providing a method for breeding a novel plant, Brassica campestris L. ssp. chinensis Hongkyeongchae which comprises the following steps of; obtaining a hybrid F₁ by crossing Brassica campestris var. chinensis as a seed parent with Brassica koreana Lee var. redleaf; obtaining a F₂ plant by selecting strains and individuals from the interspecific hybrids; obtaining a B₁F₁ plant having the shape of conventional Brassica campestris var. chinensis and showing red color by back crossing Brassica campestris var. chinensis and the F₂ plant; obtaining a plurality of red Brassica campestris var. chinensis from the progenies obtained by back crossing various strains of B₁F₁ plants selected from the obtained plants with Brassica campestris var. chinensis, and carrying out strain acclimation and a self-incompatibility test, a seed productivity test and a local adaption test of a remarkable strain of a plant having the shape of conventional Brassica campestris var. chinensis and showing red color.

### ADVANTAGEOUS EFFECT

According to the present invention, the inventive novel plant, Brassica campestris L. ssp. chinensis Hongkyeongchae breeded from Brassica campestris var. Chinensis and Brassica koreana Lee var. redleaf is useful for leaf wrap, green vegetable juice, salad or Kimchi and has industrial applicability.

### DESCRIPTION OF DRAWINGS

FIG. 1 is a breeding diagram showing the process of obtaining the inventive novel plant, Brassica campestris L. ssp. chinensis Hongkyeongchae by breeding Brassica campestris var. chinensis and Brassica koreana Lee var. redleaf collected from 2000 to 2012.

FIG. 2 is a photograph showing external appearance of the inventive novel plant, Brassica campestris L. ssp. chinensis Hongkyeongchae.

FIG. 3 is a comparative photograph showing difference of the seed characteristics between seed parent Brassica campestris var. Chinensis and the inventive novel plant, Brassica campestris L. ssp. chinensis Hongkyeongchae.

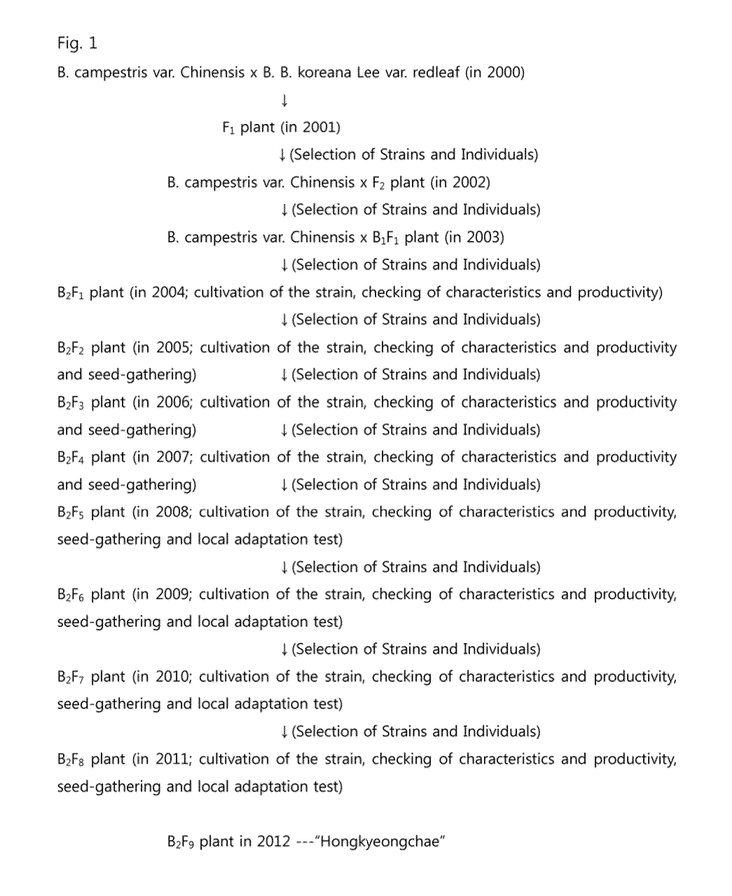

### MODE FOR INVENTION

The inventive novel plant Hongkyeongchae can be obtained by selecting a plant having the shape of conventional Brassica campestris var. Chinensis and showing red color from breeding Brassica campestris var. chinensis and Brassica koreana Lee var. redleaf. The inventive novel plant Hongkyeongchae shows characteristics as follows and is useful for leaf wrap, green vegetable juice, salad and Kimchi.

Further to the inventive breeding method, Jeokkyeongchae which has violet color could be found and it is also in the scope of the present invention.

### <Characteristics of Hongkyeongchae>

### 1. Leaf

### Shape: Oval and Round

Leafy: The outside and the inside show red color, but in case of the outside, the density is lower than that of the inside. Lower part of the leafy of Brassica koreana Lee var. redleaf is protruded and thick, whereas the inventive novel plant Hongkyeongchae is less protruded and less thick.
Leaf blade: The front and the back face are scarlet and when it is winter, they may be violet and black.
Leaf vein: It is more deep red than the leaf blade and glossy.
Margin of lead: There are neither incisions nor curves and it is simple.
Face of leaf: It is glossy.
Mesophyll: It is crispy.
2. Shape of the head: It is loose-headed.
3. Size of head at harvesting time: It is less than that of Brassica campestris var. chinensis and weighs about 30-80 grams.
4. Self-incompatibility: It is weak and it is seed vernalization type like radishs and Chinese cabbages.
5. Bolting: It is later than that of the seed parent Brassica campestris var. chinensis and late-bolting.
6. Growing rate: It is later than that of the seed parent Brassica campestris var. chinensis.
7. Color of Seeds: It is more yellowy than that of Brassica campestris var. Chinensis.

Hereafter, the present invention will be described in detail. However, it does not limit the scope of the present invention.

### Example 1

From 2000 to 2003, the present inventors abandoned an interspecific hybrid F₁ of Brassica campestris var. chinensis and Brassica koreana Lee var. redleaf for the stabilization of a hybrid embryo and obtained B₁F₁ plants by back crossing various strains of B₁F₁ plants having the shape of conventional Brassica campestris var. chinensis and showing red color. The 3₁F₁ plants were back crossed with seed parents (Brassica campestris var. chinensis) and obtained B₂F₁ plants and among them, there are a lot of Brassica campestris var. chinensis showing red color. From 2003 to 2012, the present inventors carried out strain acclimation and a self-incompatibility test, a seed productivity test and a local adaption test of a remarkable strain of a plant having the shape of conventional Brassica campestris var. chinensis and showing red color.

TABLE 1
Breeding scheme of the inventive novel plant, Brassica campestris L. ssp.
chinensis Hongkyeongchae

| Items | 00 | 01 | 02 | 03 | 04 | 05 | 06 | 07 | 08 | 09 | 10 | 11 | 12 |
|---|---|---|---|---|---|---|---|---|---|---|---|---|---|
| Collecting Data | → | | | | | | | | | | | | |
| Strain Acclimation | — | — | — | — | — | — | — | — | → | | | | |
| Incompatibility test | — | — | — | — | — | — | — | — | → | | | | |
| Making combination | — | — | — | — | → | | | | | | | | |
| Combination ability test & Selection | — | — | — | — | — | — | — | — | — | — | — | — | → |
| Productivity test | — | — | — | — | — | — | — | — | → | | | | |
| Seed test | — | — | — | — | — | — | — | — | → | | | | |
| Local ability test | | | | | | | | | | — | — | — | → |

### Example 2

### Comparison of Characteristics of Brassica campestris L. ssp. chinensis Hongkyeongchae with Conventional Brassica campestris var. Chinensis

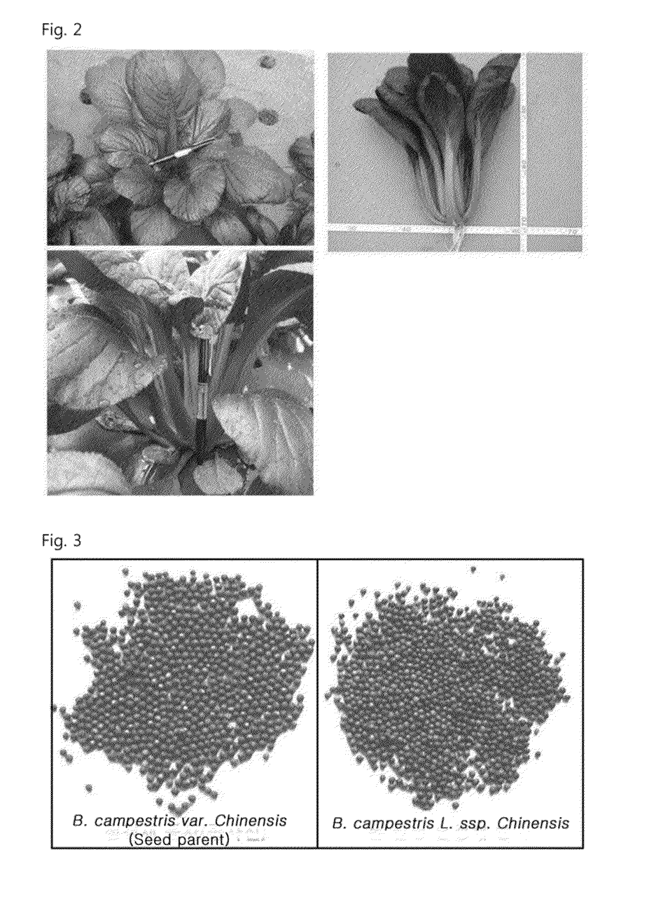

In this example, as shown in FIG. 2, the inventors examined size of the plant body, shape of the outer leaf, width and sphere of the outer leaf and compared them with those of conventional Brassica campestris var. Chinensis as control. As shown in the table 2, the height is middle, the shape of the leaf is oval and there is no leafstalk. The front and the back face of the leaf blade show deep scarlet color and it may be violet and black when it is winter. The leaf vein shows deeper red color than the leaf blade and it is glossy. Particularly, anthocyanin which is excellent antioxidant is produced in the inventive Brassica campestris L. ssp. chinensis Hongkyeongchae whereas conventional Brassica campestris var. Chinensis does not. There are neither incisions nor curves and it is simple. It is weak in self-incompatibility and it is seed vernalization type like radishs and Chinese cabbages. In addition, the seed of the inventive Brassica campestris L. ssp. chinensis Hongkyeongchae is deeper yellow than that of Brassica campestris var. Chinensis which is seed parent (Table 2, FIG. 3).

The present novel plant has been named as “Brassica campestris L. ssp. chinensis Hongkyeongchae”. Also, its English name is “Korean red pokchoi, Hongkyeongchae” and Chinese name is “ [symbol] ”.

TABLE 2
Comparison of Characteristics of Brassica campestris L. ssp. chinensis
Hongkyeongchae with conventional Brassica campestris var. Chinensis

| | | | | | | | | | | | Present | Control (Brassica |
|---|---|---|---|---|---|---|---|---|---|---|---|---|
| | | | | | | | | | | | invention | campestris var. |
| | | Phynotype | | | | | | | | | (Hongkyeongchae) | Chinensis) |
| No | Characteristics | 1 | 2 | 3 | 4 | 5 | 6 | 7 | 8 | 9 | No | No |
| 1 | Outer leaf: form | Stand | Slightly | Horizontal | | | | | | | 2 | 2 |
| | | upright | stand | | | | | | | | | |
| 2 | Plant Height | | | short | | middle | | tall | | | 5 | 5 |
| 3 | Outer leaf: | | | short | | middle | | long | | | 4 | 5 |
| | length | | | | | | | | | | | |
| 4 | Outer leaf: | | | narrow | | middle | | wide | | | 4 | 5 |
| | width | | | | | | | | | | | |
| 5 | Leaf: shape | round | Wide | Upside | Narrow | oval | Long | | | | 5 | 5 |
| | | | oval | down oval | oval | | oval | | | | | |
| 6 | Leaf: edge | blunt | round | flat | | | | | | | 2 | 2 |
| 7 | Leaf: embossing | | | weak | | middle | | strong | | | 1 | 1 |
| 8 | Leaf: size of | | | small | | Middle | | big | | | 1 | 1 |
| | embossing | | | | | | | | | | | |
| 9 | Leaf: color | scarlet | Olive | Green | Sage- | | | | | | 1 | 3 |
| | | | green | | green | | | | | | | |
| 10 | Leaf: density of color | | | light | | middle | | dark | | | 7 | 5 |
| 11 | Leaf: anthocyanin | No | | | | | | | | Yes | 9 | 1 |
| 12 | Leaf: gloss | | | Weak | | Middle | | Strong | | | 7 | 5 |
| 13 | Leafstalk: incision | No | | A little | | middle | | A lot | | Very | 1 | 1 |
| | | | | | | | | | | much | | |
| 14 | Width of main vein | Wide | | Middle | | Narrow | | | | | 3 | 2 |
| 15 | Head: size | Small | | Middle | | Big | | | | | 1 | 3 |
| 16 | Head: Weight | Light | | Middle | | Heavy | | | | | 2 | 3 |
| 17 | Seed: Color | Weak | | Middle | | Strong | | | | | 2 | 1 |
| | (Yellowness) | | | | | | | | | | | |

### INDUSTRIAL APPLICABILITY

The present invention provides a a novel plant variety, Brassica campestris L. ssp. chinensis Hongkyeongchae and a seed thereof which is useful for a leaf wrap, green vegetable juice, salad or making Kimchi and has industrial applicability.

1. A novel plant Hongkyeongchae (Brassica campestris L. ssp. Chinensis Hongkyeongchae) obtained from the seed parent of breeding Brassica campestris var. Chinensis with Brassica koreana Lee var. redleaf and performing line acclimation and having characteristics as follows:
<Characteristics of Hongkyeongchae>
1. Leaf
Shape: Oval and Round
Leafy: The outside and the inside show red color, but in case of the outside, the density is lower than that of the inside. Lower part of the leafy of Brassica koreana Lee var. redleaf is protruded and thick, whereas the inventive novel plant Hongkyeongchae is less protruded and less thick.
Leaf blade: The front and the back face are scarlet and when it is winter, they may be violet and black.
Leaf vein: It is more deep red than the leaf blade and glossy.
Margin of lead: There are neither incisions nor curves and it is simple.
Face of leaf: It has gloss.
Mesophyll: It is crispy.
2. Shape of the head: It is similar to that of Brassica campestris var. Chinensis and loose-headed.
3. Size of head at harvesting time: It is less than that of Brassica campestris var. Chinensis and weighs about 30-80 grams.
4. Self-incompatibility: It is weak and it is seed vernalization type like radishs and Chinese cabbages.
5. Bolting: It is later than that of the seed parent Brassica campestris var. Chinensis and late-bolting.
6. Growing rate: It is later than that of seed parent Brassica campestris var. Chinensis.
7. Color of Seeds: It is more yellow than that of seed parent Brassica campestris var. Chinensis.

2. A method for breeding a novel plant, Brassica campestris L. ssp. chinensis Hongkyeongchae which comprises the following steps of; obtaining a hybrid F₁ by crossing the seed parent, Brassica campestris var. chinensis with Brassica koreana Lee var. redleaf; obtaining a F₂ plant by selecting strains and individuals from the interspecific hybrids; obtaining a B₁F₁ plant having the shape of conventional Brassica campestris var. chinensis and showing red color by back crossing Brassica campestris var. chinensis and the F₂ plant; obtaining a plurality of red Brassica campestris var. chinensis from the progenies obtained by back crossing various strains of B₁F₁ plants selected from the obtained plants with Brassica campestris var. chinensis, and carrying out strain acclimation and a self-incompatibility test, a seed productivity test and a local adaption test of a remarkable strain of a plant having the shape of conventional Brassica campestris var. chinensis and showing red color.

3. A seed of a novel plant, Brassica campestris L. ssp. chinensis Hongkyeongchae (KCTC 12259BP) according to the method of claim 2 for breeding a novel plant, Brassica campestris L. ssp. chinensis Hongkyeongchae which comprises the following steps of; obtaining a hybrid F₁ by crossing the seed parent, Brassica campestris var. chinensis with Brassica koreana Lee var. redleaf; obtaining a F₇ plant by selecting strains and individuals from the interspecific hybrids; obtaining a B₁F₁ plant having the shape of conventional Brassica campestris var. chinensis and showing red color by back crossing Brassica campestris var. chinensis and the F₂ plant; obtaining a plurality of red Brassica campestris var. chinensis from the progenies obtained by back crossing various strains of B₁F₁ plants selected from the obtained plants with Brassica campestris var. chinensis, and carrying out strain acclimation and a self-incompatibility test, a seed productivity test and a local adaption test of a remarkable strain of a plant having the shape of conventional Brassica campestris var. chinensis and showing red color and showing the characteristics of claim 1.
